(19) United States
(12) Patent Application Publication
(10) Pub. No.: US 2017/0156732 A1
(43) Pub. Date: Jun. 8, 2017

(54) COORDINATED DELIVERY OF COPD TREATMENT

(71) Applicant: PneumRx, Inc., Santa Clara, CA (US)

(72) Inventors: David Lehrberg, Redwood City, CA (US); Mark L. Mathis, Fremont, CA (US); Verna Rodriguez, Santa Cruz, CA (US); Jeffrey Etter, Fremont, CA (US); Kevin Mitz, Campbell, CA (US); Christoph Steven, Marl (DE)

(73) Assignee: PneumRx, Inc., Santa Clara, CA (US)

(21) Appl. No.: 15/432,853

(22) Filed: Feb. 14, 2017

Related U.S. Application Data
(63) Continuation of application No. PCT/US2015/045514, filed on Aug. 17, 2015.
(60) Provisional application No. 62/038,058, filed on Aug. 15, 2014.

(51) Int. Cl. A61B 17/12; A61B 90/98; A61B 17/128; A61B 90/96

(52) U.S. Cl. CPC A61B 17/12104; A61B 90/96; A61B 90/98; A61B 17/12145; A61B 17/12036; A61B 17/1285; A61B 2017/00809

(57) ABSTRACT

Methods, systems and devices are disclosed for the efficient and coordinated delivery of COPD treatment to the lung(s) of a patient. A lung volume reduction system is disclosed comprising an implantable device adapted to be delivered to an airway of a patient in a constrained configuration and to change to a tissue-compressing configuration when deployed at a target zone to provide treatment to the lung airway. The invention further discloses a method of quickly and efficiently deploying the device using a single coordinated motion or signal which may be particularly useful when multiple devices are deployed at multiple target zones.

### CROSS-REFERENCES TO RELATED APPLICATIONS

The present application is a Continuation of PCT/US2015/045514 filed Aug. 17, 2015; which claims priority to and the benefit of U.S. Provisional Patent Application No. 62/038,058 filed Aug. 15, 2014; the contents of which are incorporated herein by reference in their entirety for all purposes.

### BACKGROUND OF THE INVENTION

Chronic obstructive pulmonary disease, also known as COPD, is a progressive disease that makes it difficult to breathe. COPD can cause coughing that produces large amounts of mucus, wheezing, shortness of breath, chest tightness, and other symptoms.

COPD reduces air flows in and out of the airways (i.e. trachea, bronchi and bronchioles), and is associated with the loss of structural qualities and/or support of the airways and air sacs (i.e. alveoli) damaged or destruction of the walls between many of the alveoli, the walls of the airways becoming thick and inflamed, and/or the airways producing an abundance of mucus.

COPD includes emphysema and chronic bronchitis. Many patients who have COPD have both emphysema and chronic bronchitis, and the general term “COPD” applies to both conditions. In chronic bronchitis, the lining of the airways is constantly irritated and inflamed. This causes the lining of the airways to thicken. A plethora of thick mucus forms in the airways, making it hard to breathe. In emphysema, the walls between many of the alveoli are damaged. As a result, the alveoli lose their shape and become flaccid This damage can also destroy the walls of the alveoli, leading to fewer and larger alveoli instead of many tiny alveoli. If this happens, the amount of total gas exchange in the lungs is reduced.

The medical literature describes emphysema as a chronic (long-term) lung disease that can get worse over time. Some reports indicate that emphysema is among the largest causes of mortality in the United States, affecting millions of people, with thousands of sufferers dying of the disease each year. Smoking has been identified as a major cause, but with ever increasing air pollution and other environmental factors that negatively affect pulmonary patients, the number of people affected by emphysema may be increasing.

A currently available solution for patients suffering from emphysema is a surgical procedure called Lung Volume Reduction (LVR) surgery whereby diseased lung is resected and the volume of the lung is reduced. This allows healthier lung tissue to expand into the volume previously occupied by the diseased tissue and allows the diaphragm to recover. Higher than desirable mortality and morbidity are associated with this invasive procedure. Several minimally invasive therapies have been proposed to improve the quality of life and restore lung function for patients suffering from emphysema. The underlying theory behind many of these therapeutic devices is to achieve absorptive atelectasis by preventing air from entering diseased portions of the lung, while allowing air and mucous to pass through the device out of the diseased regions. Unfortunately, collateral ventilation (interlobar and intralobar-porous flow paths that prevent complete occlusion) may prevent atelectasis, so that not all patients actually achieve the desired results. The lack of atelectasis or lung volume reduction may drastically reduce the effectiveness of such devices. Some proposed biological treatments utilize tissue engineering and/or other materials and are aimed at causing scarring at specific locations. Unfortunately, it can be difficult to control the scarring and to prevent uncontrolled proliferation of scarring. Hence, improved and/or alternative lung treatment techniques would be desirable.

One alternative and promising COPD treatment that was recently developed relies on an implant or device disposed within an airway to mechanically compress a localized portion of lung tissue, which may help restore safe and healthy tension to the remaining lung tissue. The PneumRx™ implant device may thereby provide effective treatment for COPD patients without the massive trauma of open lung volume reduction surgery, and despite collateral ventilation at the implant site that is often found in damaged COPD lung tissues. While these newly proposed devices and therapies appear to present a real improvement for many patients, as with most successes, even further improvements would be desirable. In particular, patients may benefit from deployment of several implants in each lung, and those implants may be of different sizes. While deployment of each implant is relatively straightforward, each implant deployment should be handled with care. Unfortunately, the total cumulative time to deploy all the implants for a patient can be somewhat longer than would be ideal to encourage rapid adoption of these promising therapies such that tens (or even hundreds) of thousands of current COPD patients can benefit.

Therefore, it would be desirable to provide improved medical devices, systems, and methods, including (for example) to provide lung implant devices, systems and methods that facilitate deploy in a timely, efficient and safe manner. It would also be particularly beneficial to simplify the overall deployment procedure, particularly when multiple devices of different sizes are deployed to multiple target zones in the lung(s).

### BRIEF SUMMARY OF THE INVENTION

Certain embodiments of the invention relate generally to medical devices, systems, and methods, with exemplary embodiments being particularly useful for the treatment of COPD in one or both lungs of a patient by introducing elongate implant structures into a target airway axial region of the lung airway system. The target axial region may or may not include branches, and the implants can optionally be released within the airway to allow the implant to bend so that the implant compresses adjacent lung tissue. Multiple implants may each locally compress adjacent lung tissue from within the airways of the lung, thereby providing beneficial tension in other (often, though not always healthier) portions of the lung. At least some of the implants may be deployed within the lung sequentially, with one or more of the implants being partially deployed by holding a proximal end of the implant at a fixed location and withdrawing a surrounding catheter or other implant structure relative to the implant, and thereafter, the implant being partially deployed by moving the proximal end of the implant distally during compression of the lung tissue so as to limit axial loading between the implant and the airway. The implants within a single lung may have different lengths, and the desired axial movement distances of the implants and support structures may vary with the implant lengths. By providing a linkage configured to effect coordinated movements of the implants and associated implant support structure(s) in response to a simple input movement or command, the total time for treatment of the patient may he sufficient shortened to increase utilization of these beneficial therapies.

In accordance with a first aspect, the coordinated delivery of COPD treatments include a method for treating a lung with lung tissue and an airway. The airway has a target zone. The method comprises advancing a distal end of a delivery system into the airway of the lung so that the distal end is adjacent a distal portion of the target zone. The delivery system comprises a flexible implant support, a linkage, an input, and a first implant. A distal portion of the first implant is engaged to the lung tissue along the distal portion of the airway target zone. The delivery system is actuated using an input movement of the input. The linkage couples axial movement of the first implant to the support so that, in response to the first input movement, the delivery system moves a proximal portion of the first implant distally along the airway relative to the lung tissue (thereby defining a first delivery system output movement) and moves a distal end of the implant support proximally along the airway target zone relative to the lung tissue, in coordination with the first delivery output movement, (thereby defining a second delivery system output movement). The coordinated first and second output movements are performed so that at least some part of the first implant that is disposed proximal of the distal portion of the first implant progressively recovers from a constrained configuration toward a tissue-compressing configuration. The first implant is deployed from the support, and the deployed implant locally compresses the lung tissue adjacent the airway target zone.

Typically, the first input movement comprises moving the input continuously in an input movement direction and by an input displacement distance. The first delivery system output movement may comprise distal movement of at least a portion of the first implant by a first distance. The second delivery system output movement may comprise proximal movement of the support by a second distance. The delivery system optionally coordinates the first and second distances so as to reduce and/or inhibit axial loading between the first implant and the airway such that an axial recovery displacement of the proximal portion of the first implant upon the deployment of the first implant from the implant support is within a desired range. The first distance may be about the same as the second distance, or may differ from the second distance significantly from the second distance, with the shorter of the distances optionally differing by no more than 90%, no more than 70% or no more than 50% from the longer, and in most cases being at least 5% of the longer (with the first distance being longer for some embodiments, and the second distance being longer for other embodiments).

The first and second distances may be shorter or longer than a length of the airway target zone. As can be understood with reference to U.S. Pat. No. 8,632,605 entitled “Elongate Lung Volume Reduction Devices, Systems, and Method” (incorporated herein by reference), deployment of implants may optionally include measurement of a target zone of an airway, such as by measuring a length between a distal end of a bronchoscope and a distal end of a catheter or guidewire having an appropriate diameter (corresponding to that of the implant) that has been advanced through the airway until the distal end of the catheter or guidewire engages with the airway, which may provide tactile feedback to the operator. An implant can be selected based on the measured target zone length, with the selected implant often being significantly longer than the target zone, such as more than 10% longer, more than 30% longer, and often being very roughly 100% longer (measured relative to the target zone length). For example, if the target zone has a measured length of about 60 mm an implant having a straightened length of about 125 mm may be selected; a target zone of about 85 mm may be appropriate for an implant with a straightened length of about 140 mm. Similarly comparing the lengths of the first and second distances associated with the first and second delivery system output movements, respectively, to the target zone length—the first distance may be between 5% and 200% of the target zone length, often being between 50% and 170% of the target zone length, and ideally being about (very roughly) 125% of the target zone length; the second distance may be between 5% and 200% of the target zone length, often being between 50% and 125% of the target zone length, and ideally being about (very roughly) 100% of the target zone length.

In exemplary embodiments, the method further comprises actuating the delivery system to deploy a second implant. The delivery system can move a proximal portion of the second implant distally toward another airway target zone to define a third delivery system output movement. The distal end of the implant support may move proximally along the other airway target zone relative to the lung tissue, in coordination with the third delivery output movement, to define a fourth delivery system output movement. The length of the second implant may differ from the length of the first implant. The coordinated third and fourth delivery system output movements have third and fourth distances, respectively, and the third and fourth distance will optionally differ from the first and second distances, respectively, in correlation with the lengths of the implants so that a portion of the second implant proximal to the distal portion of the second implant progressively recovers from a restrained configuration toward a tissue-compressing configuration and such that an axial recovery displacement of the proximal portion of the second implant is within the desired range upon deployment of the second implant from the implant support.

The delivery system typically comprises a tubular access device and the linkage may be included among a plurality or set of alternative selectable linkages coupleable to the access device adjacent a proximal end of the delivery system. The linkages are optionally each configured to effect coordinated movements of the distal end of the delivery system. An associated sequential series of implants can each have an associated implant length. For example, one of the linkages may comprise a first rack axially coupleable to the first implant. A second rack is axially coupleable to the support. A pinion is disposed between and engages with both racks so that rotation of the pinion induces opposed first and second output motions. Alternatively, one of the linkages may comprise a pulley and a flexible tether having a first end axially coupleable with the first implant and a second end axially coupleable with the support. The tether engages the pulley between the ends so that movement of the tether induces the opposed first and second output motions.

In exemplary embodiments, a first powered actuator moves the first implant relative to a base with the first delivery system output movement with a first command signal. A second powered actuator moves the support relative to the base with the second delivery system output movement per a second command signal. A processor, coupled to the powered actuators, receives a first implant signal associated with a size of the first implant and transmits the command signals in response to the implant signal. The processor transmits alternative command signals to the actuators in response to a second implant signal associated with a size of the second implant. The first and second implant signals are optionally generated with sensors indicating proximity and/or automated data code readers, such as those using radiofrequency identification (RFID) codes, barcodes, two-dimensional (2D) matrix codes, QR codes, magnetic codes, or spectral barcodes associated with the implants.

The support optionally comprises a delivery catheter having a lumen receiving the first implant and constraining the implant in a straighter configuration. A shaft may be releasably axially affixed to the implant, and a bronchoscope having a working channel can receive the catheter and a viewing surface near the distal end. A base of the linkage may be axially constrained relative to the bronchoscope and the lung tissue during the first and second output delivery movements.

The distal portion of the first implant can optionally be initially engaged with the lung tissue by moving the implant distally relative to the implant support and the lung tissue by an initial engagement distance that defines an initial engagement movement. The initial engagement distance may be in a range from about 10 mm to about 40 mm, optionally being from about 20 mm to about 30 mm. The delivery system induces the initial engagement movement in response to the first input movement.

In many embodiments, the distal portion of the first implant has a distal arc with an axial arc length. The arc length may optionally define a bend of over 45 or 90 degrees, often being 180 degrees or more, and ideally defining more than ¾ of a loop and/or less than 1 ½ loops (with the implant optionally extending proximally along another contiguous arc length having a bend in the same direction—such as in a helical coil—or in a different plane). The distal portion of the first implant can be deployed to couple with the lung tissue with a distal portion deployment movement by proximally retracting the implant support relative to the first implant, while maintaining an axial location of the first implant relative to the lung tissue, by a distance corresponding to the axial arc length so that the distal arc laterally engages the adjacent airway. The axial arc length may a range from about 20 mm to about 75 mm The distal portion deployment movement is often induced by the first input movement.

Coordinated proximal pulling of the support (for the second output movement) and distal advancement of the implant proximal end will (for the first output movement) often be performed simultaneously or substantially simultaneous and with concurrent or overlapping overall movement times (such as via alternating sequential incremental movements). The method may further comprise halting the first output movement in response to a proximal end of the first implant advancing distally beyond the bronchoscope, as shown in a remote imaging modality (such as fluoroscopy, ultrasound, magnetic resonance imaging, or the like) or in an image acquired by the bronchoscope. Release of the implant can be completed by proximally retracting the implant support proximally of the implant. The shaft can be recapturably detached from the implant.

In exemplary embodiments, the delivery system comprises a processor coupleable with a nonvolatile computer-readable storage medium having data associated with actuation of the delivery system. This facilitates adjusting axial lengths of the various deployment motions disclosed herein according to the length of an implant, particularly when that implant is selected from among a plurality or set of alternative implants having differing lengths. The processor may receive signals indicating a length, lot number, unique identification number, or other characteristics of the implant, optionally via sensors indicating proximity and/or automated data code readers, such as those associated with an RFID tag, bar code, QR code, or the like affixed to the implant or its packaging.

A second embodiment of the invention provides a delivery system for treating a lung having lung tissue and an airway. The airway has a target zone. The delivery system comprises an elongate flexible implant support extending between a proximal end and a distal end. The distal end is configured to be advanced distally into the airway of the lung so that the distal end is adjacent a distal portion of the target zone. An input is moveable to define an input movement. A first implant is releasably supportable by the implant support. The first implant has an elongate body extending between a proximal implant portion and a distal implant portion. The first implant is configured for deployment along the target zone from an axial configuration extending along the implant support to a deployed configuration to compress lung tissue adjacent the target zone. A linkage couples the input to the proximal end of the implant support and to the first implant so that, when the distal portion of the implant engages the lung tissue along the distal portion of the airway target zone and in response to the input movement, the linkage moves a proximal end of the first implant distally along the airway relative to the lung tissue to define a first delivery system output movement. The implant support moves proximally along the airway target zone relative to the lung tissue, in coordination with the first delivery output movement, to define a second delivery system output movement. The first and second output movements are coordinated so that the portion of the first implant proximal of the distal implant portion progressively recovers from the axial configuration toward the deployed configuration.

The linkage may optionally be configured to effect the first and second delivery system output movement when the first input movement comprises moving the input continuously in an input movement direction and by an input displacement distance. In other embodiments, a simple series of input motions may be used, such as repeatedly pushing a button or the like.

The first delivery system output movement optionally comprises distal movement of the implant by a first distance. The second delivery system output movement optionally comprises proximal movement of the support by a second distance. The delivery system optionally coordinates the first and second distances so as to reduce and/or inhibit axial loading between the first implant and the tissue such that an axial recovery displacement of the proximal portion of the first implant upon the deployment of the first implant from the implant support is within a desired range. The shorter of the first and second distance optionally differs from the longer of the first and second distance by less than 90%, 70%, or 50%.

The delivery system optionally comprises a second implant. The delivery system is optionally configured to move a proximal portion of the second implant distally toward another airway target zone to define a third delivery system output movement. A distal end of the implant support is optionally moved proximally along the other airway target zone relative to the lung tissue, in coordination with the third delivery output movement to define a fourth delivery system output movement. A length of the second implant optionally differs from a length of the first implant. The coordinated third and fourth delivery system output movements may have third and fourth distances, respectively, and the third and fourth distances optionally differ from the first and second distances, respectively, in correlation with the lengths of the implants so that the portion of the second implant proximal of the distal implant portion progressively recovers from a constrained configuration toward a tissue-compressing configuration such that an axial recovery displacement of the proximal portion of the second implant upon deployment (or detachment) of the second implant from the implant support is within the desired range.

The delivery system optionally comprises a tubular access device and the linkage is optionally among a plurality of alternative selectable linkages coupled to the access device adjacent a proximal end of the delivery system. The linkages are optionally each configured to effect associated coordinated movements of the distal end of the delivery system and a sequential series of implants having an associated implant length. One of the linkages of the delivery system optionally comprises a first rack axially coupleable to the first implant, a second rack axially coupleable to the support, and a pinion disposed between and engaging both racks so that rotation of the pinion induces the opposed first and second output motions.

One of the linkages of the delivery system optionally comprises a pulley and a flexible tether having a first end axially coupleable with the first implant and a second end axially coupleable with the support. The tether optionally engages the pulley between the ends so that movement of the tether induces the opposed first and second output motions.

The linkage of the delivery system optionally comprises a first powered actuator operably coupled with the first implant to move the first implant relative to a base with the first delivery system output movement with a first command signal. A second powered actuator is optionally operably coupled with the implant support to move the implant support relative to the base with the second delivery system output movement per a second command signal. A processor is optionally coupled to the powered actuators. The processor is optionally configured to receive a first implant signal associated with a size of the first implant and transmit the command signals in response to the implant signal. The processor optionally transmits alternative command signals to the actuators in response to a second implant signal associated with a size of the second implant. The first and second implant signals are optionally generated using sensors indicating proximity and/or automated data code readers, such as those associated with radiofrequency identification (RFID) codes, barcodes, two-dimensional (2D) matrix codes, QR codes, magnetic codes, or spectral barcodes associated with the implants.

The support optionally comprises a delivery catheter having a lumen for receiving the first implant and constraining the implant in a straighter/delivery configuration therein. A shaft is optionally advanced within the lumen and releasably engaged with the implant. In alternative embodiments, the implant may have a lumen that receives the support therein so as to constrain the implant. A bronchoscope for use with or in the system optionally has a working channel and an image capture device. The working lumen receives the delivery catheter therethrough. A base of the linkage is optionally axially constrainable relative to the bronchoscope and the lung tissue during the first and second output delivery movements.

The linkage is optionally configured to initially engage the distal portion of the first implant with the lung tissue by moving the implant distally relative to the implant support and the lung tissue by an initial engagement distance so as to define an initial engagement movement, wherein the initial engagement distance is optionally in a range from about 10 mm to about 40 mm. The delivery system optionally induces the initial engagement movement in response to the first input movement. The distal portion of the first implant optionally has a distal arc with an axial arc length. The linkage is optionally configured to couple the distal portion of the first implant to the lung tissue with a distal portion engagement movement by proximally retracting the implant support relative to the first implant by a distance corresponding to the axial arc length, while maintaining an axial location of the first implant relative to the lung tissue, so that the distal arc laterally engages the adjacent airway. The axial arc length is optionally in a range from about 20 mm to about 75 mm.

The linkage is optionally configured so that the distal portion engagement movement is induced by first input movement. The linkage is optionally configured to halt the first output movement in response to a proximal end of the first implant advancing distally beyond the bronchoscope, to proximally retract the implant support proximally of the implant and/or to recapturably detach the shaft from the implant. A processor is optionally coupled with a nonvolatile computer-readable storage medium. The processor is optionally configured to record data associated with actuation of the delivery system on the medium.

These and other features, aspects, and advantages of various embodiments of the invention will become better understood with regard to the following description, appended claims, accompanying drawings and abstract.

### BRIEF DESCRIPTION OF THE DRAWINGS

FIGS. 1A-1C illustrate the anatomy of the human respiratory system;

FIGS. 2A and 2B illustrates a bronchoscope;

FIG. 3 illustrates a bronchoscope in combination with a delivery device for a lung volume reduction device according to embodiments of the invention;

FIG. 4A-4C illustrate a device implanted within the kings;

FIG. 5 illustrates a device configuration;

FIG. 6 illustrates an alternative device configuration;

FIG. 6A shows a plurality of different implants of differing lengths;

FIG. 7 schematically shows a lung that has an upper lobe treated by deployment of a plurality of devices;

FIGS. 8A-8B illustrate how a distance between device ends is reduced when deforming from a delivery configuration to a deployed configuration;

FIG. 9A illustrates a system for delivering an implant into an airway with coordinated delivery motions in response to actuation of a trigger handle or rotation of a knob using a gear mechanism;

FIG. 9B illustrates a system for delivering an implant into an airway with coordinated delivery motions using a slider and pulley mechanism;

FIGS. 10A-10D illustrates a method for deployment of an implant into an airway using coordinated motions per a processor and a powered deployment system;

FIG. 11 is a flow chart schematically illustrating a method for delivery an implant into an airway using coordinated motions output by a deployment system;

FIGS. 11A-11C illustrate coordinated motions for a deployment sequence performed using a handle and a gear mechanism;

FIGS. 12A-12C illustrate coordinated motions for a deployment sequence performed using a rotation knob and a gear mechanism;

FIGS. 13A and 13B illustrate a system for performing coordination motion of a deployment sequence using a bar and a pulley mechanism;

FIG. 14 is a flow chart showing method steps for treating a lung of a patient according to embodiments of the invention; and

FIG. 15 shows a handle system for maintaining an axial location of one or more elements of an implant deployment system relative to tissues of a patient.

### DETAILED DESCRIPTION OF THE INVENTION

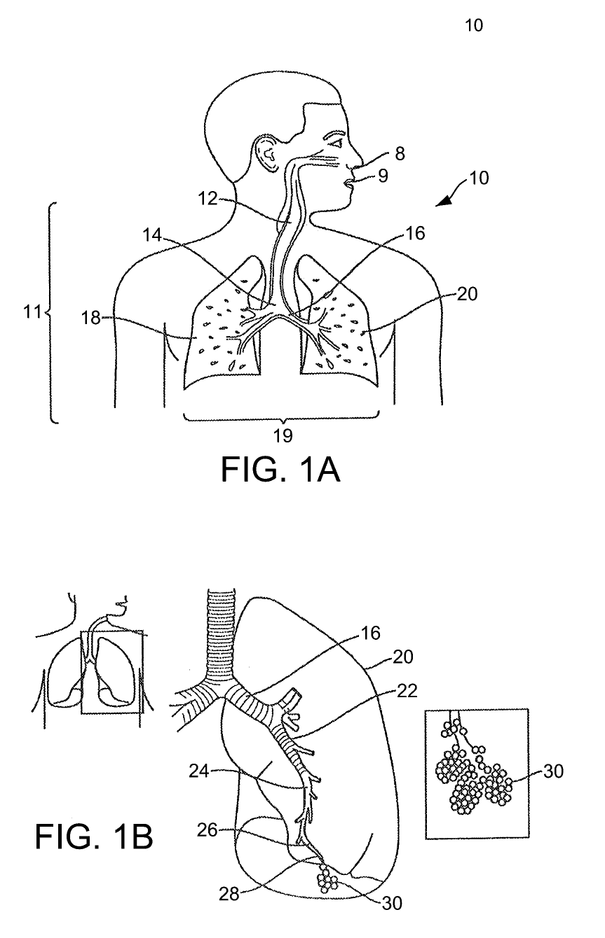

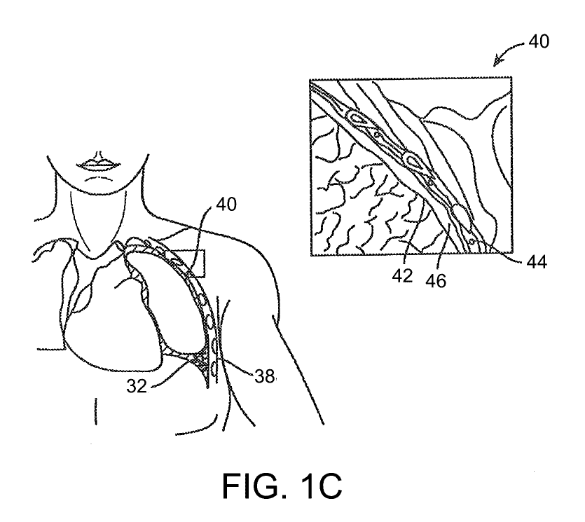

By way of background and to provide context for the invention, FIG. 1A illustrates the respiratory system 10 located primarily within a thoracic cavity 11. This description of anatomy and physiology is provided in order to facilitate an understanding of the invention. Persons of skill in the art, will appreciate that the scope and nature of the invention is not limited by the anatomy discussion provided. Further, it will be appreciated there can be variations in anatomical characteristics of an individual, as a result of a variety of factors, which are not described herein. The respiratory system 10 includes the trachea 12, which brings air from the nose 8 or mouth 9 into the right primary bronchus 14 and the left primary bronchus 16. From the right primary bronchus 14 the air enters the right lung from the left primary bronchus 16 the air enters the left lung 20. The right lung 18 and the left lung 20, together comprise the lungs 19.

The left lung 20 is comprised of only two lobes while the right lung 18 is comprised of three lobes, in part to provide space for the heart typically located in the left side of the thoracic cavity 11, also referred to as the chest cavity.

As shown in more detail in FIG. 1B, the primary bronchus, e.g. left primary bronchus 16, that leads into the lung, e.g. left lung 20, branches into secondary bronchus 22, and then further into tertiary bronchus 24, and still further into bronchioles 26, the terminal bronchiole 28 and finally the alveoli 30. As can be seen in FIG. 1C, the pleural cavity 38 is the space between the lungs and the chest wall. The pleural cavity 38 protects the lungs 19 and allows the lungs to move during breathing. The pleura 40 defines the pleural cavity 38 and consists of two layers, the visceral pleurae 42 and the parietal pleurae 44, with a thin layer of pleural fluid therebetween. The space occupied by the pleural fluid is referred to as the pleural space 46. Each of the two pleurae layers 42, 44, are comprised of very porous mesenchymal serous membranes through which small amounts of interstitial fluid transude continually into the pleural space 46. The total amount of fluid in the pleural space 46 is typically slight. Under normal conditions, excess fluid is typically pumped out of the pleural space 46 by the lymphatic vessels.

The lungs 19 are described in literature as an elastic structure that floats within the thoracic cavity 11. The thin layer of pleural fluid that surrounds the lungs 19 lubricates the movement of the lungs within the thoracic cavity 11. Suction of excess fluid from the pleural space 46 into the lymphatic channels maintains a slight suction between the visceral pleural surface of the lung pleura 42 and the parietal pleural surface of the thoracic cavity 44. This slight suction creates a negative pressure that keeps the lungs 19 inflated and floating within the thoracic cavity 11. Without the negative pressure, the lungs 19 collapse like a balloon and expel air through the trachea 12. Thus, the natural process of breathing out is almost entirely passive because of the elastic recoil of the lungs 19 and chest cage structures. As a result of this physiological arrangement, when the pleura 42, 44 is breached, the negative pressure that keeps the lungs 19 in a suspended condition disappears and the lungs 19 collapse from e elastic recoil effect.

When fully expanded, the lungs 19 completely fill the pleural cavity 38 and the parietal pleurae 44 and visceral pleurae 42 come into contact. During the process of expansion and contraction with the inhaling and exhaling of air, the lungs 19 slide back and forth within the pleural cavity 38. The movement within the pleural cavity 38 is facilitated by the thin layer of mucoid fluid that lies in the pleural space 46 between the parietal pleurae 44 and visceral pleurae 42. As discussed above, when the air sacs in the lungs are damaged 32, such as is the case with emphysema, it is hard to breathe. Thus, isolating the damaged air sacs to improve the elastic structure of the lung improves breathing.

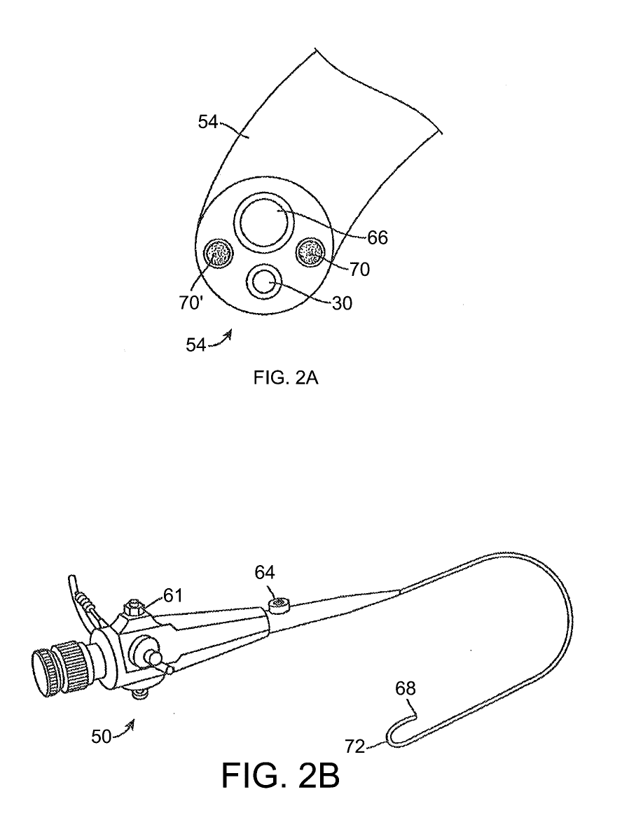

A conventional flexible bronchoscope is described in U.S. Pat. No. 4,880,015 to Nierman for Biopsy Forceps. As shown in FIGS. 2A and 2B, bronchoscope 50 can be configured to be of any suitable length, for example, measuring 790 mm in length. The bronchoscope 50 can further be configured from two main parts, a working head and an insertion tube 54. The working head 52 contains an eyepiece; an ocular lens with a diopter adjusting ring; attachments for the suction tubing and a suction valve 61 and for the cold halogen light source; and an access port or biopsy inlet 64, through which various devices and fluids can be passed into the working channel 66 and out the distal end of the bronchoscope. The working head is attached to the insertion tube, which typically measures 580 mm in length and 6.3 mm in diameter. The insertion tube can be configured to contain fiber optic bundles (which terminate in the objective lens 30 at the distal tip 68), two light guides 70, 70′ and the working channel 66. The distal end of the bronchoscope has the ability to bend 72 anterior and posterior only, with the exact angle of deflection depending on the instrument used. A common range of bending is from 160 degrees forward to 90 degrees backward, for a total of 250 degrees. Bending is controlled by the operator by adjusting an angle lock lever and angulation lever on the working head. See also, U.S. Patent Pub. 2005/0288550 A1 to Mathis, entitled Lung Access Device; US 2005/0288549 A1 to Mathis, entitled Guided Access to Lung Tissue; and US 2010/0070050 A1 to Mathis, entitled Enhanced Efficacy Lung Volume Reduction Devices, Methods and Systems, for example. All publications and patent applications mentioned in this specification are herein incorporated by reference to the same extent as if each individual publication or patent application was specifically and individually indicated to be incorporated by reference.

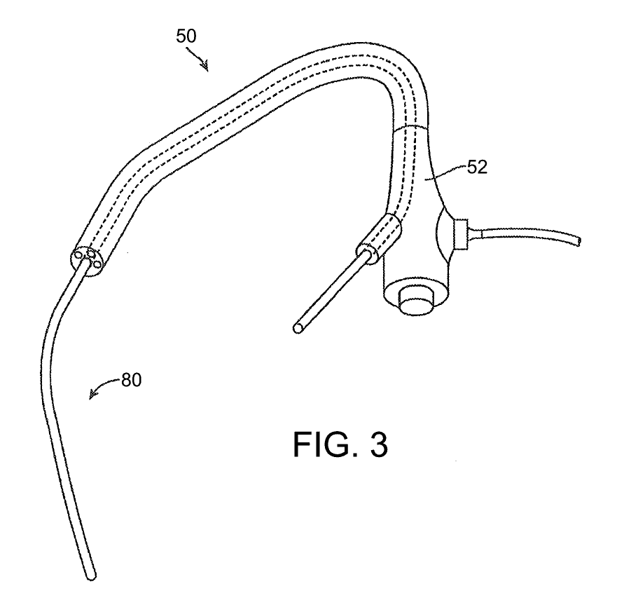

FIG. 3 illustrates the use of a lung volume reduction delivery device 80 for delivering a lung volume reduction device comprising an implantable device with the bronchoscope 50. The lung volume reduction system, as described in further detail below, is adapted and configured to be delivered to a lung airway of a patient in a delivery configuration and then changed to a deployed configuration. By deploying the device, tension can be applied to the surrounding tissue which can facilitate restoration of the elastic recoil of the lung. The device is designed to be used by an interventionalist or physician. Proper deployment of coils in the lung may benefit from a sequence of motions of at least two components: a delivery forceps or other releasable axial engagement structure that determine coil position, and the constraining catheter or other support structure that holds the coil in the delivery configuration during advancement and positioning of the coil in the lung. The coil is optionally delivered by placing the bronchoscope in the lung, introducing a guidewire and delivery catheter to the airway that is to be treated so that it's about 3-5 cm from the outer layer (pleura) of the lung, loading a coil into the catheter and guiding the distal tip of the coil through the catheter so the coil can be deployed into the airway. These steps are straight forward and generally easy for the physician to perform. The remaining coil deployment steps optionally involve a sequence of movements of the forceps and delivery catheter, and which can be somewhat time-consuming (particularly when a large number of implants are to be deployed.

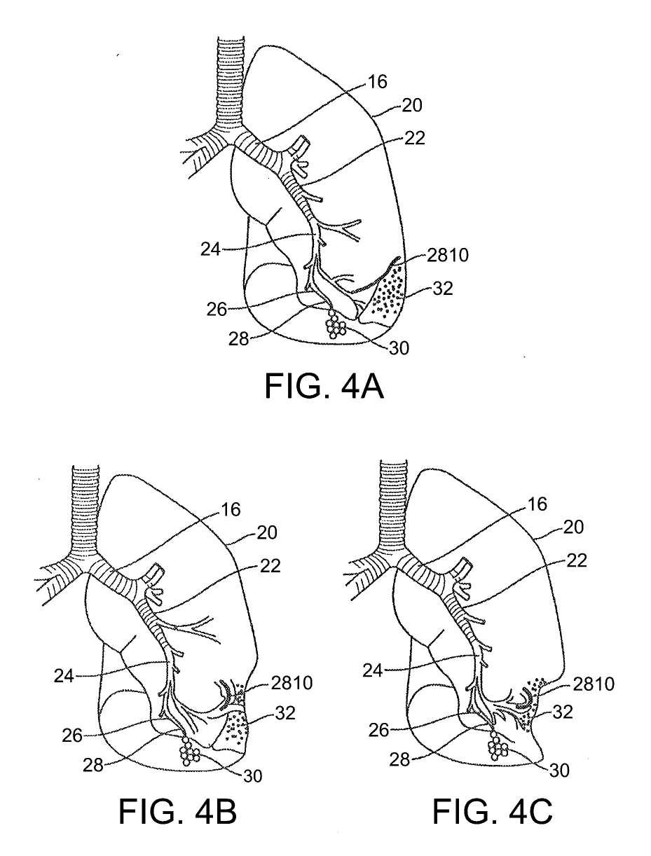

FIGS. 4A-4C schematically illustrate the effects of implanting the device within a lung. The device 2810 is advanced is a configuration where the device adapts to the anatomy of the lungs through the airways and into, for example, the bronchioles until it reaches a desired location relative to the damaged tissue 32. The device is then activated by engaging the actuation device, causing the device to curve and pull the lung tissue toward the activated device (FIG. 4B). The device continues to be activated until the lung tissue is withdrawn a desired amount, such as depicted in FIG. 4C. As will be appreciated by those skilled in the art, withdrawing the tissue can be achieved by, for example, curving and compressing a target section of lung tissue upon deployment of one of the configurable devices disclosed herein. Once activated sufficiently, the deployment device is withdrawn from the lung cavity

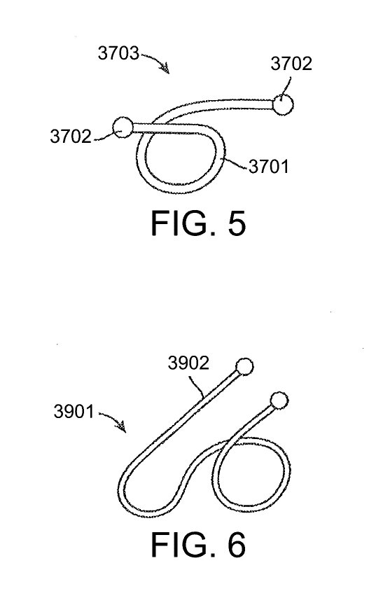

FIG. 5 shows an example of an implantable device 3703 made from Nitinol metal wire 3701. Nickel-Titanium, Titanium, stainless steel or other biocompatible metals with memory shape properties or materials with capabilities to recover after being strained 1% or more may be used to make such an implant. Additionally, plastics, carbon based composites or a combination of these materials would be suitable. The device is shaped like a French horn and can generally lie in a single plane. The ends are formed into a shape that maximizes surface area shown in the form of balls 3702 to minimize scraping or gouging lung tissue. The balls may be made by melting back a portion of the wire, however, they may be additional components that are welded, pressed or glued onto the ends of wire 3701.

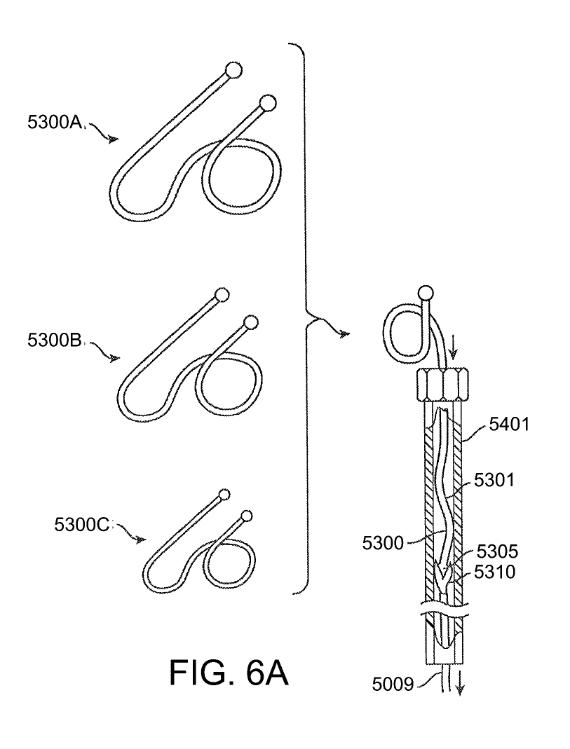

A Nitinol metallic implant, such as the one illustrated in FIGS. 5-6, may be configured to be elastic to recover to a desired shape in the body as any other type of spring would or it can be made in a configuration that may be thermally actuated to recover to a. desired shape. Nitinol can be cooled to a martensite phase or warmed to an austenite phase. In the austenite phase, the metal recovers to its programmed shape. The temperature at which the metal has fully converted to an austenite phase is known as the Af temperature (austenite final). If the metal is tuned so that the Af temperature is at body temperature or lower than body temperature, the material is considered to be elastic in the body and it will perform as a simple spring. The device can be cooled to induce a martensite phase in the metal that will make the device flexible and very easy to deliver. As the device is allowed to heat, typically due to body heat, the device will naturally recover its shape because the metal is making a transition back to an austenite phase. If the device is strained to fit through a delivery system, it may be strained enough to induce a. martensite phase also. This transformation can take place with as little as 0.1% strain. A device that is strain induced into a martensite phase will still recover to its original shape and convert back to austenite after the constraints are removed. If the device is configured with an Ar temperature that is above body temperature, the device may be heated to convert it to austenite and thermally activate its shape recovery inside the body. All of these configurations will work well to actuate the device in the patient's lung tissue. The human body temperature is considered to be 37 degrees C. in the typical human body.

FIG. 6 illustrates another implant device 3901 that is shaped in a three dimensional shape similar to the seam of a baseball. The wire is shaped so that proximal end 3902 extends somewhat straight and slightly longer than the other end. This proximal end will be the end closest to the user and the straight section will make recapture easier. If it were bent, it may be driven into the tissue making it hard to access. As will be appreciated by those skilled in the art, the devices can be configured in many different sizes and shapes including configurations included in US 2010/0070050 A1 to Mathis, entitled Enhanced Efficacy Lung Volume Reduction Devices, Methods and Systems, for example.

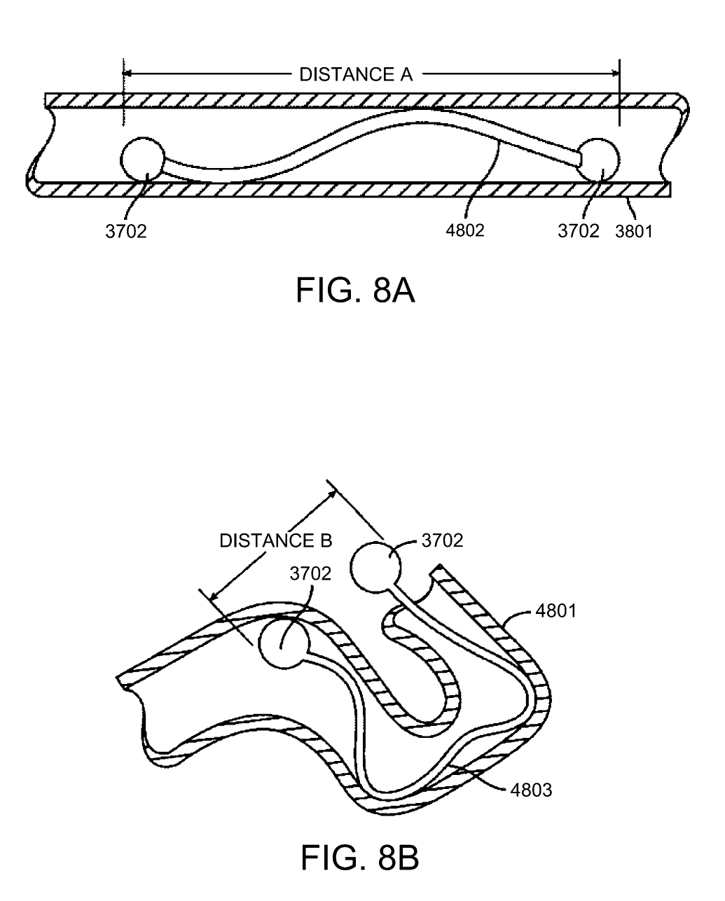

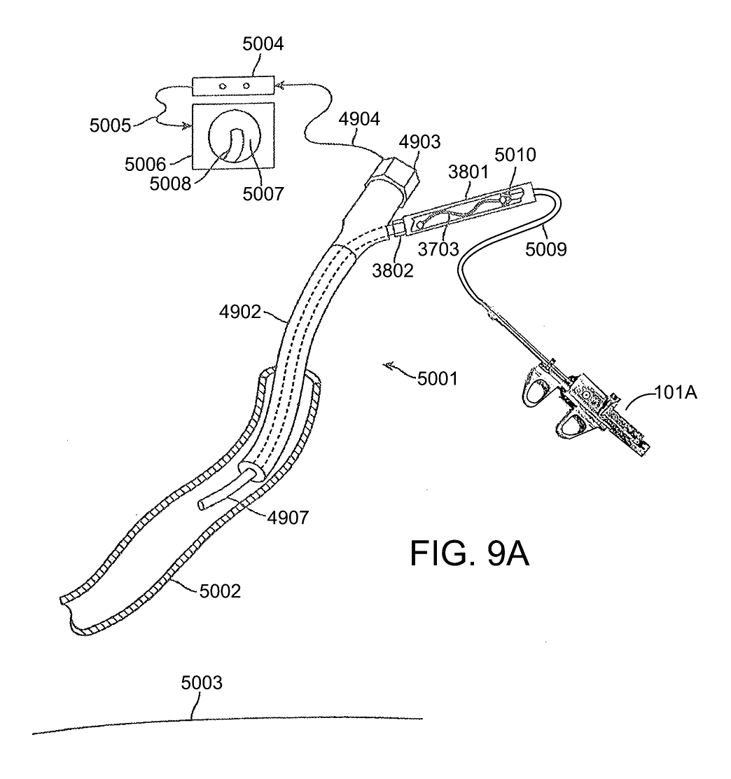

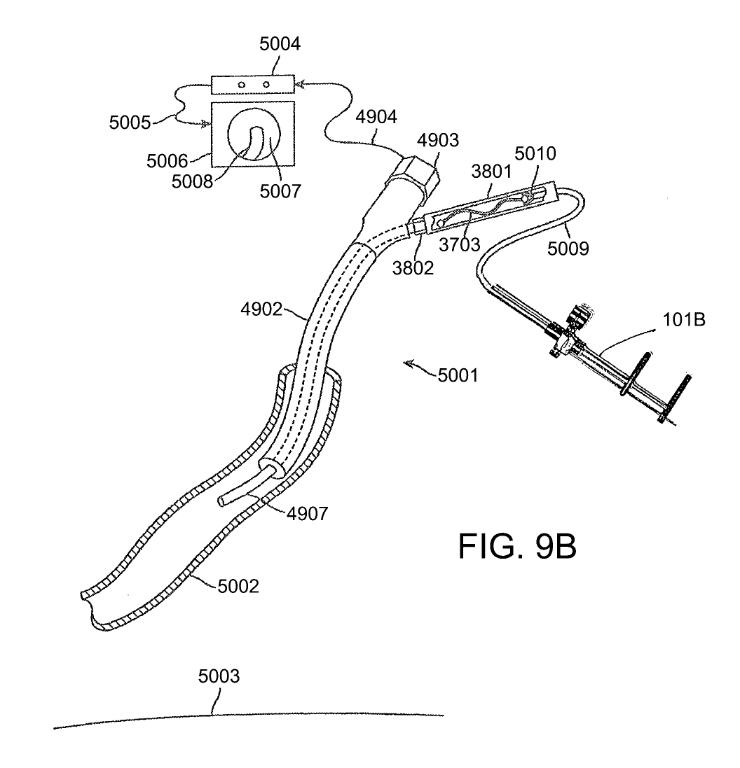

FIG. 6A shows a plurality of different implants including implants 5300A, 5300B, and 5300C. Each of these implants may have different sizes, lengths, and shapes from each other. Use of different size lung implants can be understood, for example, with reference to U.S. Pat. No. 8,632,605, entitled “Elongate Lung Volume Reduction Devices, Methods, and Systems. Using the delivery system described herein, a guidewire may be advanced to a target region near the distal end of the airway system. The guidewire may be advanced distally until further distal advancement is limited by the distal end of the guidewire being sufficiently engaged by the surrounding lumen of the airway system. Delivery catheter 4907 (see FIGS. 8 and 9) can then be advanced so that a distal end of catheter 4907 is adjacent a distal end of the guidewire. A scale or other indicia of length along the guidewire or delivery catheter can be used to measure a length of the target zone of the airway, such as between the bronchoscope and the distal end of guidewire. The desired length of the implant may be lesser, greater or about the same as the distance between the distal end of delivery catheter and distal end of the bronchoscope. To provide a desirable implant shelf life and/or a desirable deployment force for compressing tissues when using self-deploying elongate bodies (including those using resilient materials and/or using superelastic materials such as Nitinol™ or the like), it may be advantageous to store the various implants of various sizes in a relaxed state. Once the desired implant geometry or other characteristics have been identified, the selected implant may be loaded into a loading cartridge 5401 (and subsequently into the lumen of delivery catheter 4907) using pusher grasper device 5009. Pusher grasper device 5009 may be tensioned proximally and/or loading cartridge 5401 may be pushed distally so that elongate body 5301 straightens axially. The loading cartridge 5401 and implant 5300 can then be coupled to the other components of the delivery system, and the implant advanced into the airway.

In exemplary embodiments, the selected implant may have a length greater than the measured distance between the distal end of the guidewire (and hence the end of the delivery catheter) and the distal end of the scope. This can help accommodate recoil or movement of the ends of the implant toward each during delivery so as to avoid imposing excessive axial loads between the implant and tissue. Distal movement of the pusher grasper 5009 and proximal end 5305 of the implant 5300 during deployment also helps keep the proximal end 5305 of the implant 5300 within the field of view of the bronchoscope, and enhances the volume of tissue compressed by the implant, as described in the '605 patent. Exemplary implants may be more than 10% longer than the measured target airway axial region length, typically being from 10% to about 200% longer, and ideally being about 100% longer. Suitable implants may, for example, have total arc lengths of 125, 150, 175, and 200 mm, which may be appropriate for implantation in target zones of the lung having measured lengths of 60 mm, 85 mm, 110 mm, and 135 mm, respectively.

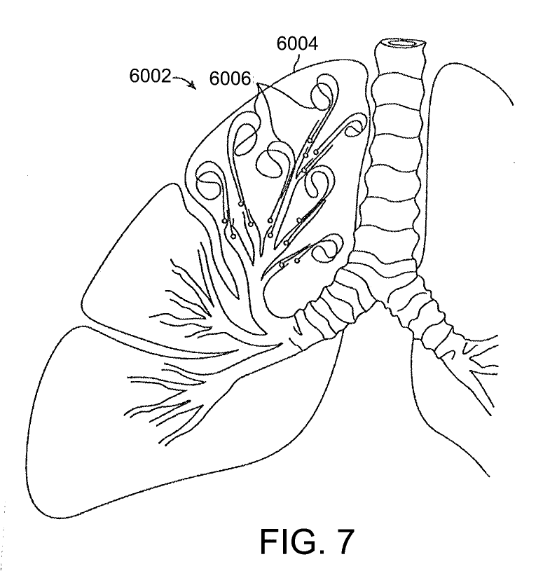

FIG. 7 is a schematic illustration of a lung 6002 that has an upper lobe 6004 treated by deployment of a plurality of implants or devices 6006, with a lobe often having between 2 and 20 devices deployed therein, optionally having between 3 and 15 devices, and in some cased between 5 and 10 devices. Different size devices will often be deployed in a patient per the local physiology per measurements from within the lung, as described above. The devices have recovered to or near their relaxed shape and the ends of the devices include locally enlarged cross-sections in the form of rounded balls so as to help the ends of the device remain in the airways they were delivered into.

FIGS. 8A and 8B illustrate how a distance between the device ends reduces with a deformation from a delivery configuration to a deployed configuration. Each of the devices depicted in FIGS. 8A and 8B are adapted and configured to impart bending force on lung tissue. The device shown in the delivery configuration 4802 in FIG. 8A is also shown in the deployed configuration 4803 in FIG. 8B. The distance A between the device ends 3702 is large while the device is constrained by the constraining cartridge device 3801. Distance A is similar when the device is constrained by a loading cartridge, catheter or bronchoscope. FIG. 8B shows the same device in a deployed configuration 4803 in an airway 4801 that has been deformed by the shape recovery of the implant device. FIG. 8B shows that the distance B between the device ends 3702 is substantially shorter after the device is deployed.

FIGS. 9A and 9B generally illustrate a delivery system 5001 that has been placed into a human lung. The bronchoscope 4902 is in an airway 5002. The scope camera 4903 is coupled to a video processor 5004 via a cable 4904. The image is processed and sent through a cable 5005 to a monitor 5006. The monitor shows a typical visual orientation on the screen 5007 of a delivery catheter image 5008 just ahead of the optical element in the scope. The distal end of the delivery catheter 4907 protrudes out of the scope in an airway 5002 where the user will place an implant device 3703. The implant 3703 is loaded into a loading cartridge 3801 that is coupled to the proximal end of the delivery catheter via locking hub connection 3802. A forceps or pusher grasper device 5009 is coupled to the proximal end of the implant 3703 with a grasper coupler 5010 that is locked to the implant. By releasably coupling the pusher to the implant device and advancing pusher/grasper device 5009, the user may advance the implant to a position in the lung in a deployed configuration. The user can survey the implant placement position and still be able to retrieve the implant back into the delivery catheter, with ease, if the delivery position is less than ideal. The device has not been delivered and the bottom surface of the lung 5003 is shown as generally flat and the airway is shown as generally straight. These may both be anatomically correct for a lung with no implant devices. If the delivery position is correct, the user may release the implant into the patient.

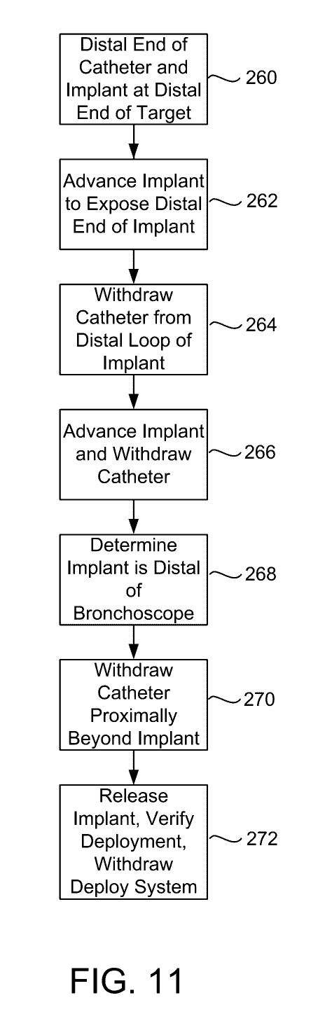

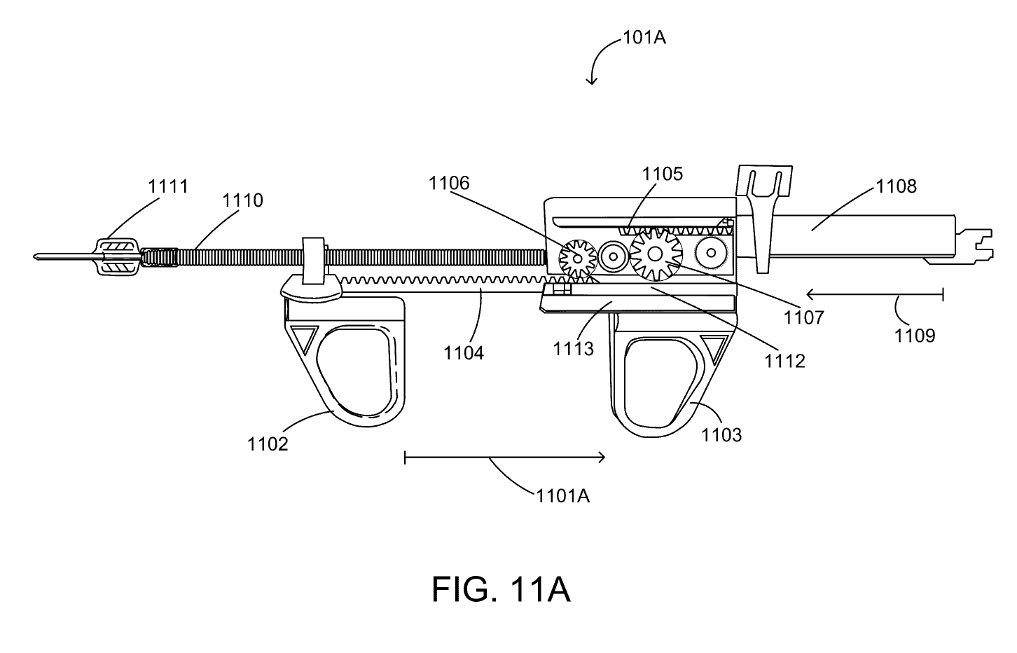

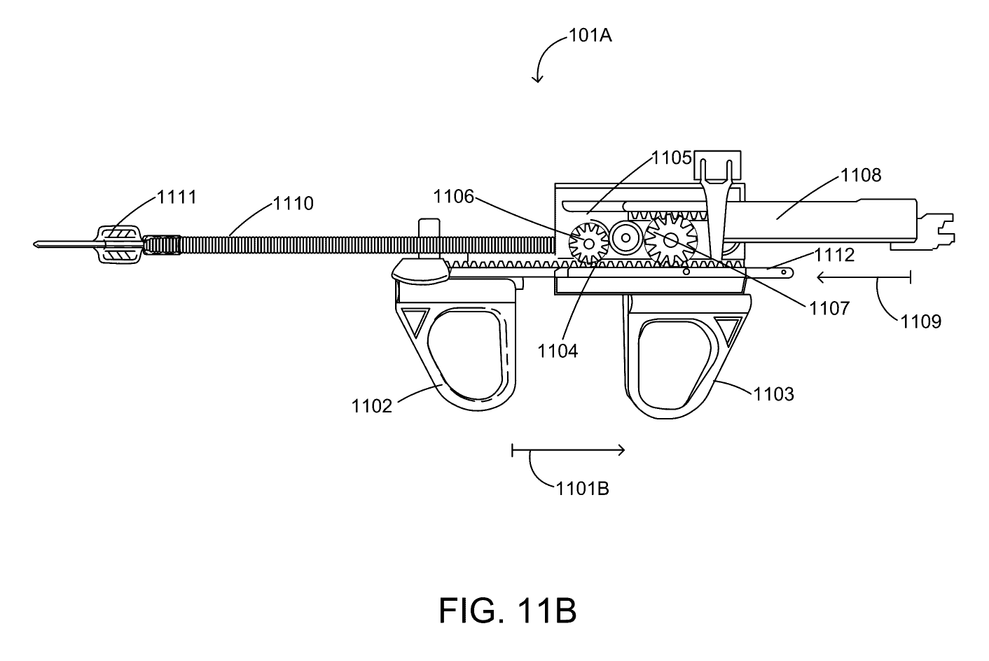

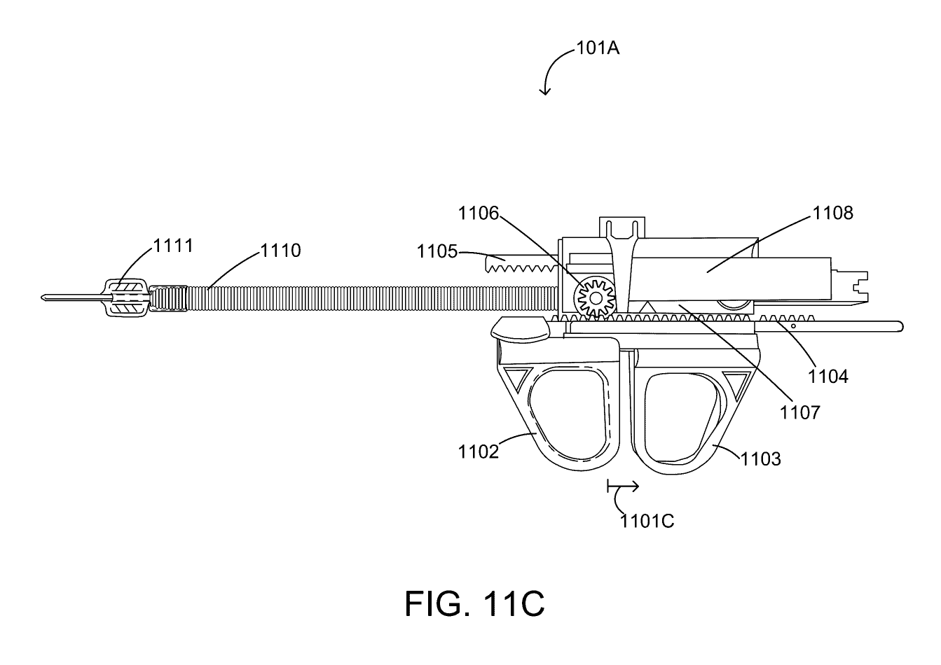

FIG. 9A shows the delivery system 5001 using a knob and/or trigger handle to actuate a geared linkage 101A to manually deploy the device 3703 as further described with respect to the following FIGS. 11A-11C.

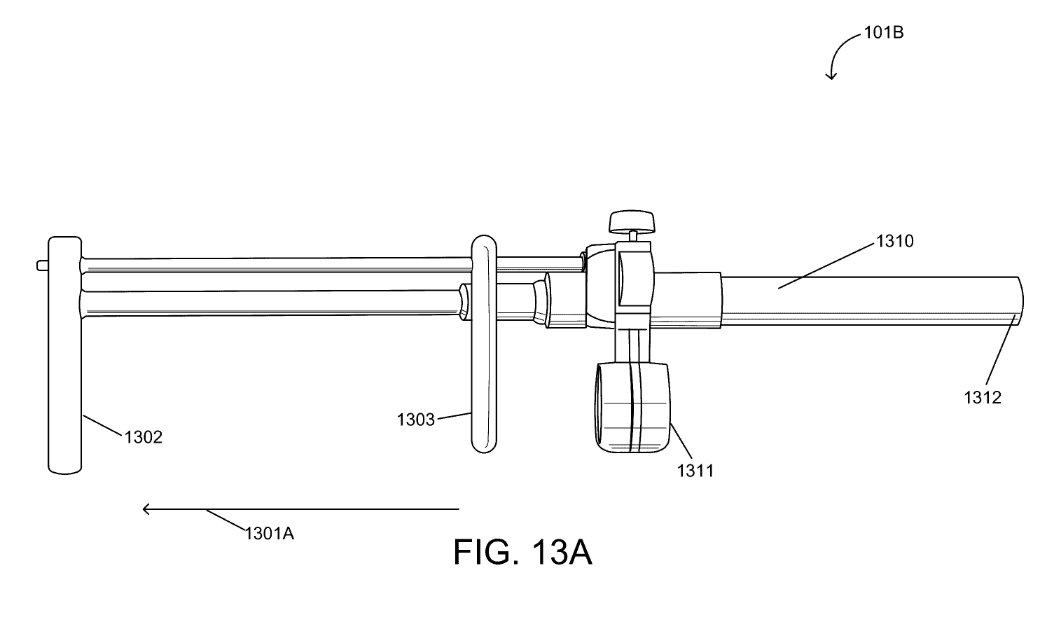

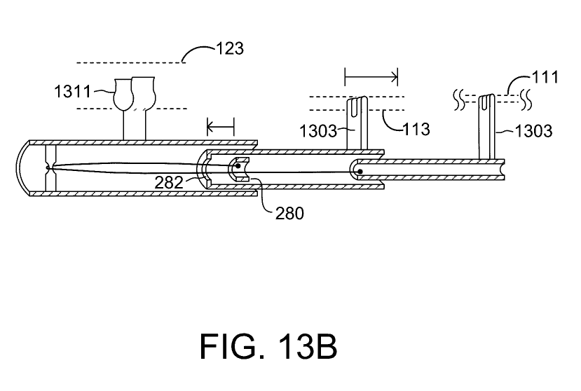

FIG. 9B generally shows a delivery system 5001 similar to that of FIG. 9A except a tether and pulley linkage 101B are used to deploy the device 3703 as further described with respect to the following FIGS. 13A and 13B.

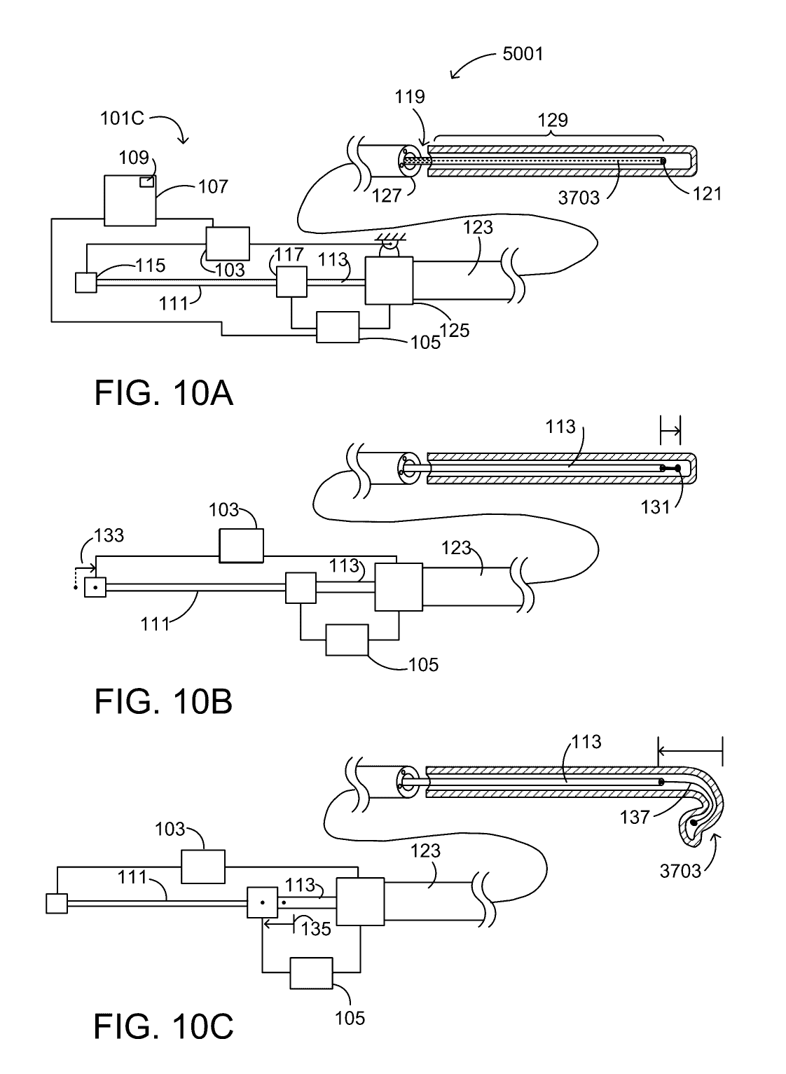

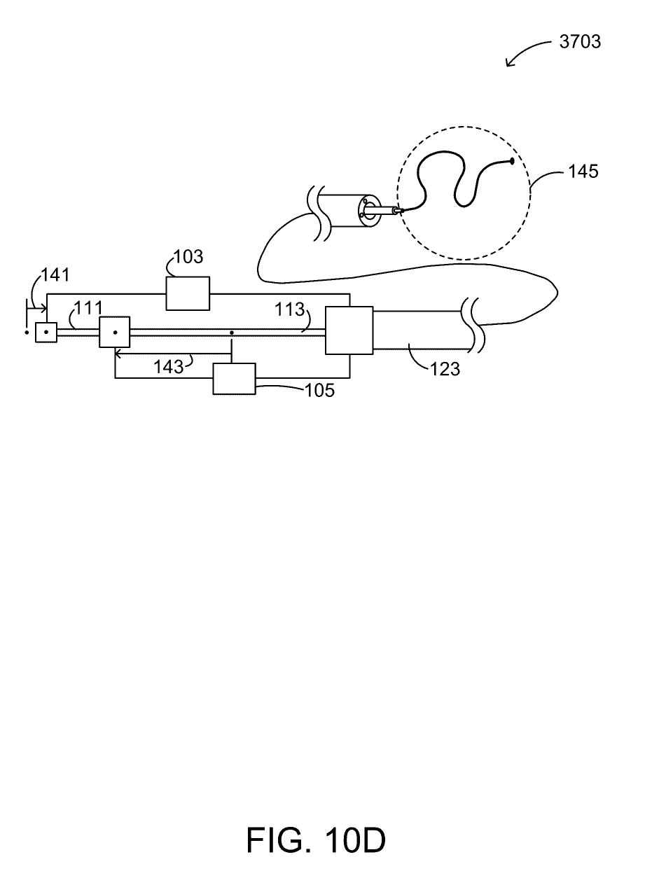

FIGS. 10A-10D generally show a delivery system 5001 similar to that of FIGS. 9A and 9B except a processor-controlled motorized linkage 101C is used to deploy device 3703 (instead of a manually actuated linkage). A motorized control may be particularly beneficial because the motions (and particularly the lengths of actuation distances) that are desired for deployment may be different for each coil length or design. Computerized control of a motor, motors or other drivers can effect axial displacement of select delivery system components, and particularly the delivery catheter and implant (via movement of the pusher/grasper device) with coordinated motions, and can also be used to tailor those coordinated motions to differing coil designs and lengths.

Tailoring of deployment displacements to differing alternatively selectable coils may optionally be provided by mechanically actuated systems, for example, by having a set of alternatively selectable deployment linkages (each having actuation displacements suitable for use with an associated implant length), by mechanical linkages with movable stops, alternatively selectable actuation elements, or the like. Motorized or other actuation systems having processor alterable actuations systems, however, may be particularly adept for patient treatments that benefit from multi-implant treatments in which implants of differing sizes are deployed. Toward that end, automated deployment system 101C includes a first motor 103 and a second motor 105, with both motors coupled to a processor 107 (not shown in all figures for simplicity). Processor 107 has or is coupled to an input 109 for receiving signals indicative of a length or other characteristics of an implant selected for deployment. The physician could identify the selected type or model of coil by entering data manually by using voice recognition or the system may detect coil data using bar coding, RFID tags, or other electronic signal(s). In response, the system may query a lookup table or other specific coil model data to determine proper coordinated deployment displacements for that particular coil product. The physician may then signal the start of the event or actuate a mechanical system. Still further implant-identifying data may be included in the coil and/or associated structures, such as the coil packaging, with the packaging or implant optionally having a bar code, being coded with magnetic strips or equipped with an RFID chip so that the coil design can be identified to the coordinated delivery system so the appropriate delivery strategy can be performed. The system may also be user programmable so that customized techniques may be employed. Regardless, input 109 may include a bar code reader, an RFID reader, a keypad or keyboard, a touch screen, a series of buttons, or the like.

An exemplary deployment using automated deployment system 101C can be understood with reference to FIGS. 10A-D and 11. Proper deployment of coils 3703 in the lung benefits from a sequence of axial motions of at least two components: the coil (via axial movement of a pusher/grasper device 111) and a delivery catheter 113. Pusher/grasper 111 and catheter 113 each have a proximal end 115, 117 and a distal end 119, 121, respectively. The coil 3703 is delivered by placing a bronchoscope 123 in the lung (segmental or sub-segmental airway), with the bronchoscope also having a proximal end 125 and a distal end 127. A guidewire and delivery catheter are introduced to the airway that is to be treated so that it's 3-5 cm from the outer layer (pleura) of the lung in step 260, thereby defining a target zone 129 between distal end 121 of delivery catheter 113 and the guidewire and distal end 127 of bronchoscope 123. The guidewire is removed and coil 3703 is loaded into delivery catheter 113 and advanced 262 using grasper/pusher 111 so that the distal tip of the coil is in alignment with the end 121 of the catheter, and adjacent a distal portion of target zone 129. Once the system is positioned, coordinated motions may optionally be initiated by actuation of input 109 in automated system 101C, and/or by manual movement of a mechanical structure that acts as an input for a mechanical actuation system. Alternatively, initial advancement of the coil and/or engagement/coupling of a distal portion of the implant to the lung tissue may be performed using discrete manual or motorized articulations of the deployment system components, with the mechanically and/or automated system providing coordinated deployment motions after one or more of these steps has been performed.

Referring now to FIG. 10B and step 262 of FIG. 11, a distal end 131 of coil 3703 may be advanced distally out of delivery catheter 113 by advancing the coil (via distal movement of pusher/grasper 111) while the delivery catheter is held at a substantially fixed location relative to the lung tissue so that a distal portion 131 the coil 3703 is pushed out of the catheter 113. When using automated deployment system 101C, a displacement 133 of pusher/grasper 111 may be effected by a motor 103 while motor 105 remains stationary, thereby holding the catheter 113 stationary relative to bronchoscope 123. The bronchoscope may generally remain or be held generally stationary relative to the patient (and/or a world reference frame), and the distal displacement of the coil may, for example, expose the distal 25 mm of the coil in the airway.

Referring now to FIG. 10C and step 264 of FIG. 11, the deployment system may pull delivery catheter 113 proximally relative to bronchoscope 123 by a displacement 135 while the coil 3703 and pusher/grasper 111 remains in a fixed position. Optionally, the catheter may be moved proximally until the first complete distal loop of the coil is exposed and unloaded into the airway. In other embodiments, a partial loop or more than a single loop may be exposed, so that the exposed arc length of the coil need not correspond to a 360 arc angle.

Referring now to FIG. 10D and step 266 of FIG. 11, deployment of much, most, or all of the length of coil 3703 may be performed by two displacements or motions: first, the pusher grasper 111 may move distally by a first displacement 141 relative to the lung tissue and/or bronchoscope 123. Second, catheter 113 may be pulled proximally by a second displacement 143 relative to the lung tissue and/or bronchoscope 123. In many embodiments, these two displacements will be effected simultaneously to further expose (deploy) the coil in the airway while advancing the forceps (and proximal end of the coil) so that the coil is allowed to recover to or toward a programmed curvilinear shape without causing excessive pulling on tissue that is distal to the coil (in other words, pushing the coil in so it can bend and shorten without causing too much tension and stress on tissue in the lung, while imposing a safe and therapeutic compression to a region or volume of lung tissue 145 adjacent the target zone of the airway).

Referring now to FIG. 11, delivery of the proximal portion of the coil out of the bronchoscope can be determined 268 by visualizing the tip of the forceps exposed out of the end of the bronchoscope under fluoroscopy, using the optical imaging of bronchoscope 123, or the like. Optionally, this determination can be used as a signal to halt the advancing motion of the pusher/grasper, and the remaining length of catheter 113 can be pulled proximally off of the coil 270. Pusher/grasper 111 can then be actuated so as to release the implant 272, the deployment can be verified via fluoro and optical imaging with the bronchoscope, and the remaining components of the deployment system 101C can be withdrawn from the patient or repositioned for deployment of the next implant.

Referring again to FIGS. 10A-11 some or all of the displacements of pusher/grasper 111 and delivery catheter 113 may be coordinated by processor 107 using motors 103, 105 per software or computer-readable programming instructions tangibly embodied in recording media, a memory, RAM, ROM, and/or the like of the processor. Alternative embodiments may employ hardware, firmware, or the like, and a wide variety of computer hardware and/or software architectures may be implemented. Motors 103, 105 may comprise any of a wide variety of electrical motors (such as stepper motors, DC motors, or the like), pneumatic motors, hydraulic motors, or the like, and the motors may be axially coupled to the axially movable components of the deployments system via lead screws, gears, tethers, or the like.

Another progression of deployment using a trigger handle and gear mechanism 101A is shown in FIGS. 11A, 11B and 11C. The mechanism 101A is coupled to the proximal end of the device 5009 (FIG. 9A) with a coupler 1111. The implant device 3703 is deployed at a target zone of an airway 5002. The coupler 1111 can be threaded, fitted, or otherwise attached to the device 5009 in a releasable manner. As shown in FIG. 9A, the device 5009 is coupled to the proximal end of the implant 3703 with a forceps grasper coupler 5010 that is connected to the implant using mechanism 101A. When a target site has been selected, usually by visualizing the airway 5002 with a camera 4903 and a monitor 5006, the physician advances the implant 3703 to a position in the lung in a deployed configuration as generally described in U.S. 2010/0070050 A1 to Mathis entitled Enhanced Efficacy Lung Volume Reduction Devices, Methods and Systems, for example, which is incorporated by reference herein in its entirety. As seen in FIG. 11A, mechanism 101A coordinates this deployment using a series of engageable gears (1106, 1107) to first pull the catheter when trigger handle 1102 is moved in a direction 1101A toward handle 1103 that is attached to casing 1113. The catheter is pulled until the complete distal loop of the implant 3703 is unloaded in an airway 5002 at a target zone. As trigger handle 1102 moves in direction 1101A, the teeth 1104 on a section of bar 1112 first engage gear 1106 to pull the catheter in direction 1101A. As the catheter is pulled, further movement of trigger 1102 moves the trigger 1102 closer to handle 1103. FIG. 11B shows the approximate midpoint of the movement 1101B. At this point, gear 1107 begins to contact and engage teeth 1104 (in addition to gear 1106) as bar 1112 slides in direction 1101B. The engagement of gear 1107 with teeth 1104, in turn, moves teeth 1105 in direction 1109 to push the forceps while simultaneously continuing to pull catheter in direction 1101B. Note that gear 1106 never engages teeth 1105. The distance the catheter is pulled before the forceps begin to be pushed can be reduced or increased by shortening or lengthening bar 1112, respectively.

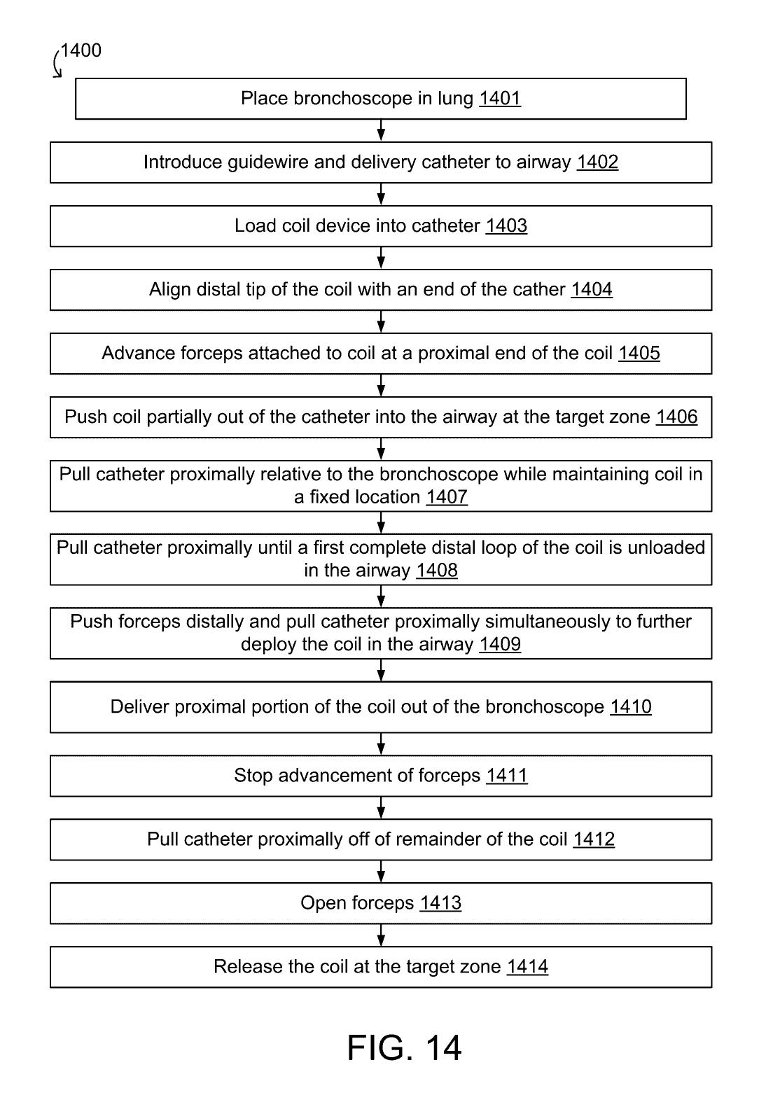

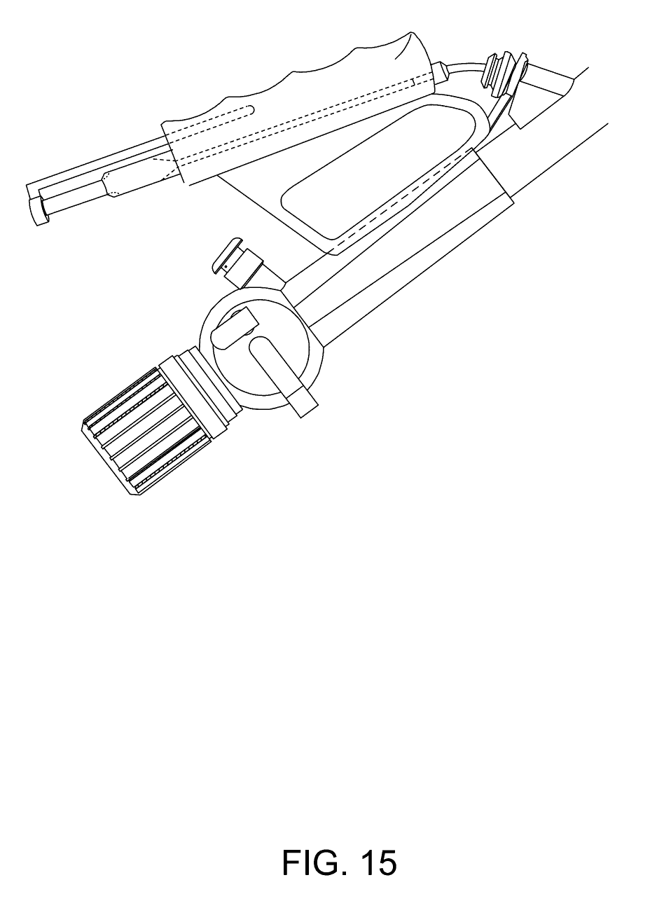

As shown in in FIG. 11C, the advancement of the forceps is stopped when trigger 1102 is pulled completely toward handle 1103 in direction 1101C. At this point, the catheter is pulled off coil and the deployment of the implant 3703 at a target zone is complete. This deployment is also depicted in method 1400, including steps 1401 through 1414, of FIG. 14, for example. This coordinated deployment makes it very efficient to implant multiple coils at various target zones (FIG. 7), with much, most, or all of each implant being deployed in response to a single coordinated motion, and with subsequent implants being deployed by loading additional implants 3703 into the cartridge 3801 (FIG. 9A) and repeating the deployment protocol.

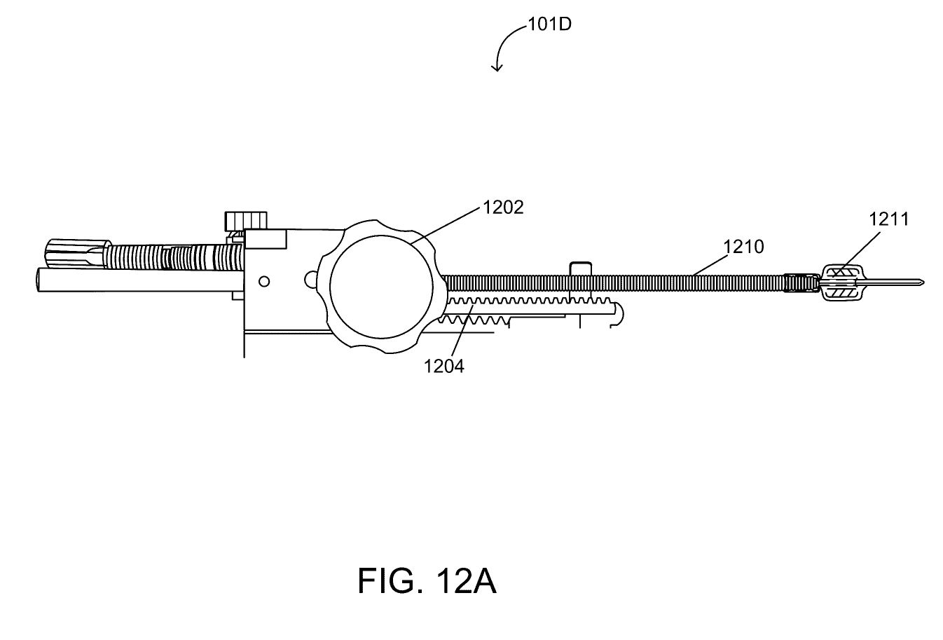

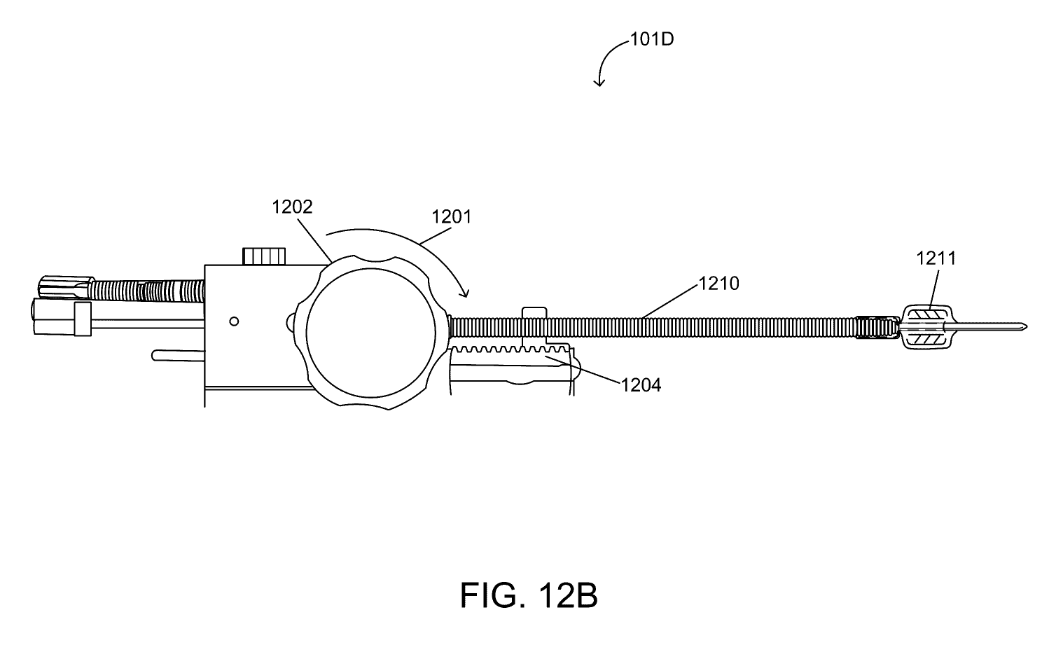

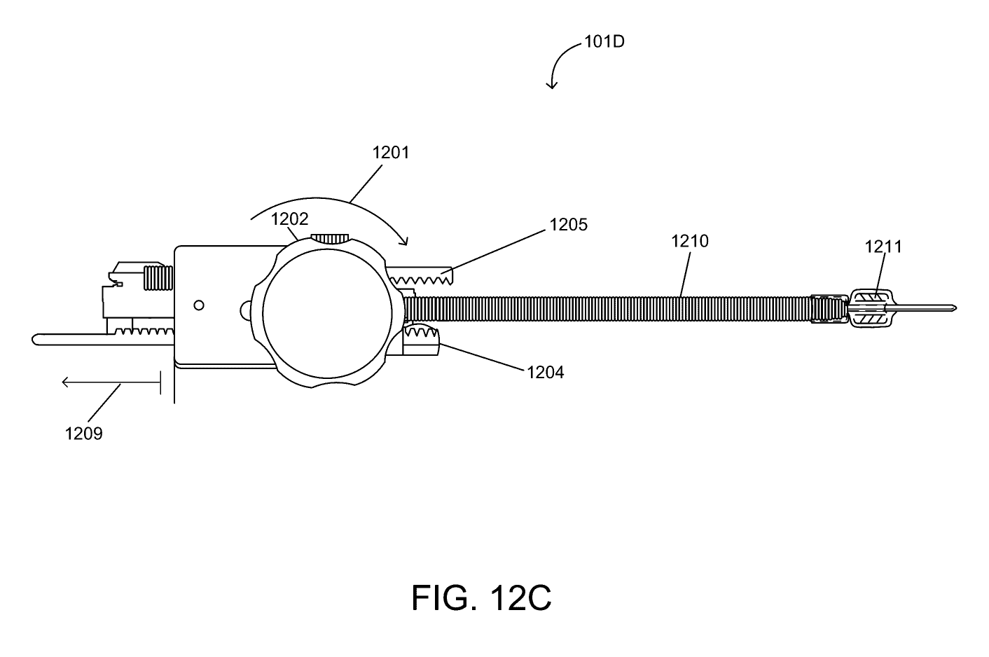

The progression of deployment using a rotational knob mechanism 101D is shown in FIGS. 12A, 12B and 12C. The mechanism 101D is coupled to the proximal end of the device 5009 (FIG. 9B) with a coupler 1211 to deploy the implant device 3703 into a target zone of an airway 5002. The coupler 1211 can be threaded, fitted, or otherwise attached to the device 5009 in a releasable manner and is coupled to the proximal end of the implant 3703 in a similar manner as previously described above with respect to FIGS. 11A through 11C.

Mechanism 101D coordinates the deployment using a series of engageable gears in a similar manner as previously described with respect to the trigger handle and gear mechanism except that a knob 1202 is moved in a direction 1201 to engage the gears (instead of using trigger handles) to move the catheter and forceps.

A related progression of deployment can be performed using a tether and pulley mechanism 101B is shown in FIGS. 13A and 13B. The mechanism 101B is coupled to the proximal end of the device 5009 (FIG. 9B) with a coupler to deploy the implant device 3703 into a target zone of an airway 5002. In this embodiment, a manual input in the form of a continuous proximal retraction of a manual input at connector 1303 (which releasably engages catheter 113) relative to bronchoscope 123 results in:

a proximal retraction of the catheter (optionally to uncover a distal loop of the coil) while a slider 280 moves distally toward a stop 282); and then

simultaneous proximal retraction of the catheter and distal advancement of pusher/grasper 111, with the simultaneous motions having the same displacement distances.

FIG. 14 is a flow chart illustrating a method 1400 for treating a lung of a patient according to embodiments of the invention. Device operation includes the step of inserting a bronchoscope into a patient's lungs 1401 and then introducing a guidewire and delivery catheter into the airway 1402 that is to be treated so that it is approximately 3-5 cm from the outer layer (pleura) of the lung. The coil device is then loaded into the catheter 1403. The distal tip of the coil is aligned with the end of the catheter 1404. A variety of methods can then be used to verify the positioning of the device to determine if the device is in the desired location. Suitable methods of verification include, for example, visualization via visualization equipment, such as fluoroscopy, CT scanning, or similar techniques. The forceps are advanced 1405 with the coil attached at the proximal end. These initial steps are relatively straight forward for the physician to perform.

The remaining coil deployment steps can be challenging and complicated yet they are also important to the efficacy and safety of the treatment. Proper deployment of coils in the lung require a sequence of motions with two main components (the delivery forceps that effect coil position and the constraining catheter that holds the coil straight).

The remaining steps to deploy the coil may be performed under guidance of fluoroscopy or other means of visualization and are ideally performed by advancing the forceps so that the coil is pushed out of the catheter to expose about the first 25 mm of the coil in the airway at the target zone 1406. The catheter is then pulled out of the bronchoscope while maintaining the coil in a fixed position 1407. Continued pulling on the catheter results in the first complete distal loop of the coil exposed and unloaded into the airway 1408. The forceps are pushed and the catheter is pulled simultaneously to further expose (deploy) the coil in the airway 1409 while advancing the forceps (and proximal end of the coil) so that the coil is allowed to recover to a programmed curvilinear shape without causing excessive pulling on tissue that is distal to the coil (e.g. push the coil in so it can shorten without causing too much tension and stress on tissue in the lung). When the proximal portion of the coil is delivered out of the bronchoscope 1410, then the advancing motion of the forceps should stop 1411 and the remaining length of catheter should be pulled off of the coil 1412. These steps can be visualized by sighting the tip of the forceps exposed out of the end of the bronchoscope under fluoroscopy, for example. The forceps can be opened 1413 and the coil is released at the target zone in the lung 1414. The aforementioned series of steps may deliver an elongate length of coil in a safe manor with beneficial treatment effect on the target zone of the lung tissue.

The bronchoscope, forceps and catheter can be mechanically coupled and all the steps to deploy the coil can be enabled with a single motion or signal. The motion can be a single slider, pulling of a single lever like a large trigger or handle, rotation of a knob, the act of pressing a button to actuate a motor, solenoid, pneumatic actuator, hydraulic actuator or other means to deliver force to drive a mechanism that moves the components in a coordinated way to perform the steps that are required. The signal could be and electronic or a verbal command that is recognized by the system to start to perform the coordinated motions.

Coordinating the motions can done by providing a mechanism that engages the components as a function of relative displacement (like moving a pinion along that engages a rack at the proper position). This would allow the catheter to be pulled and then simultaneous motion of the catheter and forceps can occur. Cables can be used the same way so that a cable end block can engage another component at a given displacement. A single pinion between two racks will force a condition of opposite motions as is needed with the catheter and forceps.

It is further contemplated that a query system could be employed to determine what deployment strategy was used during procedures. This system may assist with case complaint investigations or in the event of a safety issue that may occur, for example. Means of downloading data or making queries may include Wi-Fi or cabled communication to a computer, modem, phone communication system, magnetic sensors, visual screen reads, self-printing etc.

Although embodiments of the invention have been described in considerable detail with reference to certain preferred versions thereof, other embodiments are possible. Therefore, the spirit and scope of the appended claims should not be limited to the descriptions of the embodiments above.

What is claimed is:

1. A method for treating a lung having lung tissue and an airway, the airway having a target zone, the method comprising:
advancing a distal end of a delivery system into the airway of the lung so that the distal end is adjacent a distal portion of the target zone, the delivery system comprising a flexible implant support, a linkage, an input, and a first implant;
engaging a distal portion of the first implant to the lung tissue along the distal portion of the airway target zone;
actuating the delivery system using an input movement of the input;
wherein the linkage couples axial movement of the first implant to the support so that, in response to the first input movement, the delivery system:
moves a proximal portion of the first implant distally along the airway relative to the lung tissue so as to define a first delivery system output movement;
moves a distal end of the implant support proximally along the airway target zone relative to the lung tissue, in coordination with the first delivery output movement, so as to define a second delivery system output movement;
wherein the coordinated first and second output movements are performed so that a portion of the first implant proximal to the distal implant portion progressively recovers from a constrained configuration toward a tissue-compressing configuration; and
deploying the first implant from the support, the deployed implant locally compressing the lung tissue adjacent the airway target zone.

2. The method of claim 1, wherein the first input movement comprises moving the input continuously in an input movement direction and by an input displacement distance.

3. The method of claim 2, wherein the first delivery system output movement comprises distal movement of the implant by a first distance, wherein the second delivery system output movement comprises proximal movement of the support by a second distance, and wherein the delivery system coordinates the first and second distances so as to reduce axial loading between the first implant and the lung tissue such that an axial recovery displacement of the proximal portion of the first implant upon the deployment of the first implant from the implant support is within a desired range.

4. The method of claim 3, wherein the shorter of the first distance and the second distance differs from the longer of the first distance and the second distance by less than 90%.

5. The method of claim 3, further comprising:
actuating the delivery system so as to deploy a second implant, wherein the delivery system:
moves a proximal portion of the second implant distally toward another airway target zone so as to define a third delivery system output movement;
moves a distal end of the implant support proximally along the other airway target zone relative to the lung tissue, in coordination with the third delivery output movement, so as to define a fourth delivery system output movement;
wherein a length of the second implant differs from a length of the first implant, wherein the coordinated third and fourth delivery system output movements have third and fourth distances, respectively, and the third and fourth distances differ from the first and second distances, respectively, in correlation with the lengths of the implants so that a portion of the second implant proximal to the distal implant portion progressively recovers from a constrained configuration toward a tissue-compressing configuration such that an axial recovery displacement of the proximal portion of the second implant upon deployment of the second implant from the implant support is within the desired range.

6. The method of claim 5, wherein the delivery system comprises a tubular access device and the linkage is among a plurality of alternative selectable linkages coupleable to the access device adjacent a proximal end of the delivery system, the linkages each configured to effect coordinated movements of the distal end of the delivery system and an associated sequential series of implants, the associated implants having an associated implant length.

7. The method of claim 6, wherein one of the linkages comprises a first rack axially coupleable to the first implant, a second rack axially coupleable to the support, and a pinion disposed between and engaging both racks so that rotation of the pinion induces opposed first and second output motions.

8. The method of claim 6, wherein one of the linkages comprises a pulley and a flexible tether having a first end axially coupleable with the first implant and a second end axially coupleable with the support, the tether engaging the pulley between the ends so that movement of the tether induces the opposed first and second output motions.

9. The method of claim 5, wherein a first powered actuator moves the first implant relative to a base with the first delivery system output movement per a first command signal, wherein a second powered actuator moves the support relative to the base with the second delivery system output movement per a second command signal, and wherein a processor is coupled to the powered actuators, the processor receiving a first implant signal associated with a size of the first implant and transmitting the command signals in response to the implant signal, and wherein the processor transmits alternative command signals to the actuators in response to a second implant signal associated with a size of the second implant.

10. The method of claim 9, wherein the first and second implant signals are generated using radiofrequency identification (RFID) codes, barcodes, 2D matrix codes, QR codes, magnetic codes, or spectral barcodes associated with the implants.

11. The method of claim 1, wherein the support comprises a delivery catheter having a lumen receiving the first implant and constraining the implant in a straighter configuration, a shaft releasably axially affixed to the implant, and a bronchoscope having a working channel receiving the catheter therethrough and a viewing surface near the distal end, wherein a base of the linkage is axially constrained relative to the bronchoscope and the lung tissue during the first and second output delivery movements.

12. The method of claim 11, further comprising initially engaging the distal portion of the first implant with the lung tissue by moving the implant distally relative to the implant support and the lung tissue by an initial engagement distance so as to define an initial engagement movement, wherein the initial engagement distance is in a range from about 10 mm to about 40 mm.

13. The method of claim 12, wherein the delivery system induces the initial engagement movement in response to the first input movement.

14. The method of claim 12, wherein the distal portion of the first implant has a distal arc with an axial arc length, and wherein the distal portion of the first implant is coupled to the lung tissue with a distal portion deployment movement by proximally retracting the implant support relative to the first implant, while maintaining an axial location of the first implant relative to the lung tissue, by a distance corresponding to the axial arc length so that the distal arc laterally engages the adjacent airway, the axial arc length in a range from about 20 mm to about 75 mm.

15. The method of claim 14, wherein the distal portion deployment movement is induced by the first input movement.

16. The method of claim 12, further comprising halting the first output movement in response to a proximal end of the first implant advancing distally beyond the bronchoscope, as shown in an image acquired by the bronchoscope, proximally retracting the implant support proximally of the implant, and recapturably detaching the shaft from the implant.

17. The method of claim 11, wherein the delivery system comprises a processor coupleable with a nonvolatile computer-readable storage medium, and further comprising recording data associated with actuation of the delivery system on the medium.

18. A delivery system for treating a lung having lung tissue and an airway, the airway having a target zone, the delivery system comprising:
an elongate flexible implant support extending between a proximal end and a distal end, the distal end configured to be advanced distally into the airway of the lung so that the distal end is adjacent a distal portion of the target zone;
an input moveable so as to define an input movement;
a first implant releasably supportable by the implant support, the first implant having an elongate body extending between a proximal implant portion and a distal implant portion and configured for deployment along the target zone from an axial configuration extending along the implant support to a deployed configuration so as to compress lung tissue adjacent the target zone;
a linkage coupling the input to the proximal end of the implant support and to the first implant so that, when the distal portion of the implant engages the lung tissue along the distal portion of the airway target zone and in response to the input movement, the linkage:
moves a proximal end of the first implant distally along the airway relative to the lung tissue so as to define a first delivery system output movement;
moves the implant support proximally along the airway target zone relative to the lung tissue, in coordination with the first delivery output movement, so as to define a second delivery system output movement;
coordinates the first and second output movements so that a portion of the first implant proximal to the distal implant portion progressively recovers from the axial configuration toward the deployed configuration.

19. The delivery system of claim 18, wherein the linkage is configured to effect the first and second delivery system output movement when the first input movement comprises moving the input continuously in an input movement direction and by an input displacement distance.

20. The delivery system of claim 19, wherein the first delivery system output movement comprises distal movement of the implant by a first distance, wherein the second delivery system output movement comprises proximal movement of the support by a second distance, and wherein the delivery system coordinates the first and second distances so as to reduce axial loading between the first implant and the lung tissue such that an axial recovery displacement of the proximal portion of the first implant upon the deployment of the first implant from the implant support is within a desired range.

21. The delivery system of claim 20, wherein the shorter of the distances differs from the longer of the distances by less than 90%.

22. The delivery system of claim 21, further comprising a second implant, wherein the delivery system is configured to:
move a proximal portion of the second implant distally toward another airway target zone so as to define a third delivery system output movement;
move a distal end of the delivery system proximally along the other airway target zone relative to the lung tissue, in coordination with the third delivery output movement, so as to define a fourth delivery system output movement;
wherein a length of the second implant differs from a length of the first implant, wherein the coordinated third and fourth delivery system output movements have third and fourth distances, respectively, and the third and fourth distances differ from the first and second distances, respectively, in correlation with the lengths of the implants so that a portion of the second implant proximal to the distal implant portion progressively recovers from a constrained configuration toward a tissue-compressing configuration such that an axial recovery displacement of the proximal portion of the second implant upon deployment of the second implant from the implant support is within the desired range.

23. The delivery system of claim 22, wherein the delivery system comprises a tubular access device and the linkage is among a plurality of alternative selectable linkages coupleable to the access device adjacent a proximal end of the delivery system, the linkages each configured to effect associated coordinated movements of the distal end of the delivery system and a sequential series of implants having an associated implant length.

24. The delivery system of claim 23, wherein one of the linkages comprises a first rack axially coupleable to the first implant, a second rack axially coupleable to the support, and a pinion disposed between and engaging both racks so that rotation of the pinion induces the opposed first and second output motions.

25. The delivery system of claim 23, wherein one of the linkages comprises a pulley and a flexible tether having a first end axially coupleable with the first implant and a second end axially coupleable with the support, the tether engaging the pulley between the ends so that movement of the tether induces the opposed first and second output motions.

26. The delivery system of claim 22, wherein the linkage comprises:
a first powered actuator operably coupleable with the first implant so as to move the first implant relative to a base with the first delivery system output movement per a first command signal;
a second powered actuator operably coupleable with the implant support so as to move the implant support relative to the base with the second delivery system output movement per a second command signal; and
a processor is coupled to the powered actuators, the processor configured to receive a first implant signal associated with a size of the first implant and transmit the command signals in response to the implant signal;
wherein the processor transmits alternative command signals to the actuators in response to a second implant signal associated with a size of the second implant.

27. The delivery system of claim 26, wherein the first and second implant signals are generated using radiofrequency identification (RFID) codes, barcodes, 2D matrix codes, QR codes, magnetic codes, or spectral barcodes associated with the implants.

28. The delivery system of claim 18, wherein the support comprises a delivery catheter having a lumen for receiving the first implant and constraining the implant in a straighter configuration therein, a shaft advanceable within the lumen and releasably axially affixed to the implant, and a bronchoscope having a working channel and an image capture device, the working lumen receiving the delivery catheter therethrough, wherein a base of the linkage is axially constrainable relative to the bronchoscope and the lung tissue during the first and second output delivery movements.

29. The delivery system of claim 18, wherein the linkage is configured to initially engage the distal portion of the first implant with the lung tissue by moving the implant distally relative to the implant support and the lung tissue by an initial engagement distance so as to define an initial engagement movement, wherein the initial engagement distance is in a range from about 10 mm to about 40 mm.

30. The delivery system of claim 29, wherein the delivery system induces the initial engagement movement in response to the first input movement.

31. The delivery system of claim 29, wherein the distal portion of the first implant has a distal arc with an axial arc length, and wherein the linkage is configured to engage the distal portion of the first implant to the lung tissue with a distal portion engagement movement by proximally retracting the implant support relative to the first implant, while maintaining an axial location of the first implant relative to the lung tissue, by a distance corresponding to the axial arc length so that the distal arc laterally engages the adjacent airway, the axial arc length in a range from about 20 mm to about 75 mm.

32. The delivery system of claim 31, wherein the linkage is configured so that the distal portion engagement movement is induced by first input movement.

33. The delivery system of claim 29, wherein the linkage is configured to halt the first output movement in response to a proximal end of the first implant advancing distally beyond the bronchoscope, to proximally retract the implant support proximally of the implant, and/or recapturably detach the shaft from the implant.

34. The delivery system of claim 11, further comprising a processor coupleable with a nonvolatile computer-readable storage medium, wherein the processor is configured to record data associated with actuation of the delivery system on the medium.
